(19) United States
(12) Patent Application Publication
(10) Pub. No.: US 2023/0112768 A1
(43) Pub. Date: Apr. 13, 2023

(54) CANNULA ASSEMBLY, IN PARTICULAR FOR DRAWING LIQUID FROM A BODY

(71) Applicant: Greiner Bio-One GmbH, Kremsmuenster (AT)

(72) Inventors: Christian RATHNER, Sattledt (AT); Gerhard STRASSER, Kremsmuenster (AT); Georg KOFLER, Inzersdorf im Kremstal (AT)

(73) Assignee: Greiner Bio-One GmbH, Kremsmuenster (AT)

(21) Appl. No.: 17/800,370

(22) PCT Filed: Mar. 4, 2021
(86) PCT No.: PCT/AT2021/060072

§ 371 Date: Aug. 17, 2022

(30) Foreign Application Priority Data
Mar. 9, 2020 (AT) A 50188/2020

(51) Int. Cl. A61M 25/06; A61M 5/32

(52) U.S. Cl. CPC A61M 25/0637; A61M 5/3257

(57) ABSTRACT

A cannula arrangement includes a cannula support and a cannula held thereon. A distal flow-through channel end section of a flow-through channel of the cannula support has a stepped cross-sectional shape as seen in axial section, with multiple mutually different cross-sectional dimensions decreasing in the axial direction. Each one of the cross-sectional dimensions defines its own receiving section for the respective cannula to be received therein. Each one of the cross-sectional dimensions of the receiving sections is embodied to receive the cannula with a corresponding cross-sectional cannula dimension in each case.

The invention relates to a cannula arrangement, in particular a safety cannula arrangement, which serves for removing fluid from a body and/or for supplying a fluid into a body.

WO 2016/007981 A1 of the same applicant describes a safety needle arrangement comprising a base body, a cannula support, and a cannula which is held on a distal end of the cannula support with its proximal end. The safety needle arrangement further comprises an actuating element configured as a spring, which is arranged to act between the base body and the cannula support, as well as a locking device having a plurality of first and second locking elements engaging with one another. The first locking elements are arranged in the region of a proximal end of the base body and formed by diametrically opposite projections. The second locking elements are arranged in the region of a proximal end of the cannula support and formed by diametrically opposite recesses. The safety needle arrangement has worked well in practice, and serves as the basis for the present invention. The repeatedly required tools are disadvantageous, which results in higher unit costs.

US 2007/0016148 A1 describes a further generically designed safety needle arrangement comprising a base body with a distal end and a proximal end, wherein a hole extends in the base body between the distal end of the base body and the proximal end of the base body. The safety needle arrangement further comprises a cannula support with a distal end and a proximal end, wherein a flow-through opening extends between the two ends of the cannula support. The cannula support is adjustably accommodated in the opening of the base body in the axial direction. A cannula has a distal end and a proximal end, wherein a flow channel extends between the two ends of the cannula and within it, and the proximal end of the cannula is held on the distal end of the cannula. An adjusting element is arranged acting between the base body and the cannula support, wherein the adjusting element automatically displaces the cannula support together with the cannula held thereon from a first position, in which the cannula projects beyond the distal end of the base body, into a second position, in which at least the distal end of the cannula is covered by the base body. A locking device has a plurality of first and second locking elements. The locking device determines the first relative position of the cannula support together with the cannula with respect to the base body when the first and second locking elements are in a mutually engaged position. The first locking elements are arranged in the region of the proximal end of the base body and the second locking elements are arranged in the region of the proximal end of the cannula support. A disadvantage of this is that after unlocking the locking device and the associated holding of the cannula support, a compressive force is exerted in the axial direction on the base body by the adjusting element, thereby pressing the base body in the direction of the puncture site. This results in an additional pain stimulus exerted on the body.

It was the object of the present invention to overcome the shortcomings of the prior art and to provide a cannula arrangement being more simple to produce and having universal combination possibilities.

This object is achieved by a cannula arrangement, in particular by a safety cannula arrangement, according to the claims.

The cannula arrangement according to the invention is used in medical technology to be able to remove a fluid from the body and/or to supply a fluid into the body.

The cannula arrangement comprises
a cannula support with a distal end and a proximal end, wherein a flow-through channel extends within the cannula support between its distal end and its proximal end, and the flow-through channel defines a distal flow-through channel end section and a proximal flow-through channel end section,
a cannula with a distal end and a proximal end, wherein a flow channel extends within the cannula between its distal end and its proximal end, and
wherein the proximal end of the cannula is received in the distal flow-through channel end section of the cannula support and is held on the cannula support, in particular glued therewith, and the cannula and the cannula support define a longitudinal axis, wherein it is further provided
that as seen in axial section, the distal flow-through channel end section, starting from the distal end of the cannula support in the direction towards the proximal end of the cannula support, has a cross-sectional shape of stepped design with a first cross-sectional dimension and at least one further cross-sectional dimension,
that each one of the cross-sectional dimensions defines an own receiving section for the respective cannula to be received therein,
that the cross-sectional dimension of each receiving section, starting from the distal end of the cannula support in the direction towards the proximal end of the cannula support, is in each case smaller than the cross-sectional dimension of the receiving section immediately upstream in the axial direction, and
that each one of the cross-sectional dimensions of the receiving sections, which differ from one another, is embodied to receive the cannula with a corresponding cross-sectional cannula dimension in each case.

The thus achieved advantage is that by the repeated embodiment of receiving sections that differ from one another in one and the same cannula support, the respective predefined cannula with the cross-sectional cannula dimension corresponding to the respective receiving section can be received and attached thereto. The individual receiving sections are adapted to the respective cross-sectional cannula dimension in terms of their respective cross-sectional dimensions. Hence, an individual receiving section adapted for this purpose is provided for each cannula and is precisely adapted to the cannula. Depending on the number of selected individual receiving sections, the production of just one equal cannula support in each case with one and the same tool (injection molding tool) is required, since depending on the requirements, the cannula with the predefined cross-sectional dimension is inserted into the receiving section provided for this purpose during assembly and is attached therein.

Furthermore, it can be advantageous if the receiving sections are embodied so as to each have a hollow-cylindrical cross-section. Thus, a good and more circumferentially uniform distribution of the adhesive for attaching the cannula to the cannula support can be achieved.

Another embodiment is characterized in that multiple, in particular three, receiving sections formed behind one another in the axial direction are provided. Depending on the selected number of receiving sections located behind one another, thus, an even more universal use can be achieved with simultaneous cost reduction.

A further possible embodiment has the features that each one of the receiving sections on its proximal end forms an axial stop for the proximal end of the respective cannula received therein in each case. Furthermore, it can be provided that the respective proximal end of the cannula is supported on the corresponding axial stop of the respective receiving section resting thereon the direction towards the proximal end of the cannula support. Hence, a good positioning preciseness in the respective receiving section, in particular in the axial direction, can be achieved for each different cross-sectional cannula dimension.

Another embodiment is characterized in that the cannula support is formed by a female Luer coupling part or a component part of a safety cannula arrangement. Hence, diverse medical support bodies can be provided for diverse cannula dimensions (diameter), wherein sufficiency can be achieved for each type with just one embodiment or an additional further embodiment.

A further preferred embodiment is characterized in that, furthermore, a base body with a distal end and a proximal end is provided, wherein a hole extends in the base body between the distal end of the base body and the proximal end of the base body, and that at least the cannula support with the cannula held thereon is received in the hole and is adjustable in the axial direction in the hole. Thus, it becomes possible to provide a safety cannula arrangement in which the cannula support with its cannula held thereon can be received and guided.

Furthermore, it may be advantageous if, further, an automatically acting adjusting element is provided, said adjusting element being arranged acting between the base body and the cannula support, and the adjusting element displaces the cannula support together with the cannula held thereon from a first position, in which first position the cannula projects beyond the distal end of the base body, into a second position, in which second position at least the distal end of the cannula is covered by the base body. Hence, an automatic relative displacement of the cannula support together with the cannula held thereon relative to the base body can be achieved. This usually occurs only after release of mutually engaged retaining elements.

Another alternative embodiment is characterized in that, furthermore, a retaining device with multiple first retaining elements and multiple second retaining elements is provided, said retaining device defining the first position of the cannula support with respect to the base body in the mutually engaged position of the first and second retaining elements, and that the first retaining elements are arranged in the region of the proximal end of the base body and the second retaining elements are arranged in the region of the proximal end of the cannula support. Hence, a defined starting position and/or operational position is allowed for.

A further possible and optionally alternative embodiment has the features that, furthermore, an arresting device with multiple first and second arresting elements is provided, said arresting device defining the second position of the cannula support with respect to the base body in the mutually engaged position of the first and second arresting elements, and that the first arresting elements are arranged in the region of the proximal end of the base body and the second arresting elements are arranged in the region of the distal end of the cannula support. Hence, reuse and/or a puncture wound can be prevented after the cannula support has been moved to its second position.

In a further embodiment, it is provided that at least one relief recess is arranged or formed in the cannula support, and that, when the cannula support is in the first position, the first arresting elements project into the at least one relief recess in their undeformed position. Hence, an undeformed initial position of the first arresting elements can be achieved during storage of the cannula arrangement. Furthermore, internal tension decrease that would otherwise occur in a preformed storage position can be prevented.

Another embodiment is characterized in that, furthermore, a wing arrangement with a tube-shaped retaining body and wings projecting from the retaining body on both sides is provided, wherein the wings define a contact side which can be positioned to face a patient, and that, furthermore, a coupling unit is arranged or formed between the retaining body and the base body and the wing arrangement is held in a coupling position on the base body by means of the coupling unit. Thus, the operation and handling of the safety cannula arrangement can be facilitated for the user. Furthermore, thereby, an improved attachment to a body which is more precise in terms of position is allowed.

A further preferred embodiment is characterized in that an observation window is formed in the tube-shaped retaining body, said observation window being arranged on a side facing away from the contact side, and that in the coupling position, a protuberance projecting from the base body in the radial direction projects into the observation window. Thus, when a translucent or transparent material is selected in the area of the cannula end, it is possible for the user to easily recognize the piercing success visually.

Furthermore, it can be advantageous if the coupling unit further comprises an anti-rotation device with at least one first anti-rotation element on or in the base body and at least one second anti-rotation element cooperating therewith in or on the retaining body. By the provision of a separate anti-rotation device, a clear and rotation-resistant mounting of the wing arrangement on the base body can be achieved.

Another embodiment is characterized in that the at least one first anti-rotation element is embodied as a web and the at least one second anti-rotation element is embodied as a groove, and that the anti-rotation elements have a parallel longitudinal alignment with respect to the longitudinal axis. Hence, a positive-locking and precise positioning of the component parts being in coupled engagement can be achieved.

A further possible embodiment has the features that the coupling unit further comprises an axial securing device with at least one first axial securing element on or in the base body and at least one second axial securing element cooperating therewith in or on the retaining body. Furthermore, it can be provided that the axial securing elements, as seen in axial section, are formed approximately with a triangular cross-section, wherein a first boundary line of the triangular cross-section, as viewed in the axial push-on direction of the wing arrangement onto the base body, defines a ramp formed to rise on the side facing away from the longitudinal axis, and a second boundary line of the triangular cross-section is oriented to run in a normal plane with respect to the longitudinal axis. Thus, by holding the wing arrangement on the base body in a direction opposite to the push-on direction, a detaching movement can be prevented.

Another embodiment is characterized in that, furthermore, an in particular tube-shaped protective cover is provided and the protective cover is arranged on the distal end of the base body and is detachably held thereon, wherein the cannula projecting from the base body in distal direction in the first position is covered by the protective cover. Hence, before the first use and before the protective cover is removed from the distal end of the base body, a puncture wound can be prevented. The protective cover is preferably formed by a circumferential case wall and has a predefined axial length.

A further preferred embodiment is characterized in that at least two first longitudinal ribs and at least two second longitudinal ribs are arranged or formed distributed across the circumference on an internal surface of the protective cover facing the longitudinal axis, and that a first longitudinal rib and a second longitudinal rib are arranged alternately in the circumferential direction in each case. By the selection of the first and second longitudinal ribs, hence, a predefined formation and arrangement as seen across the inner circumference can be allowed for.

Furthermore, it can be advantageous if, in the undeformed cross-section of the protective cover, first ends of the first longitudinal ribs end at a first radial distance in front of the longitudinal axis and second ends of the second longitudinal ribs end at a second radial distance in front of the longitudinal axis, wherein the first radial distance is larger than the second radial distance. By the differently large distancing of the first ends and the second ends from the common longitudinal axis, hence, a better tolerance compensation can be achieved when the protective cover is placed on the base body. Hence, in the region of those longitudinal ribs with the largest radial distancing of their ends from the longitudinal axis, the radial prestressing force can be slightly reduced and thus a reduced widening of the cover wall in this circumferential section can be achieved.

Another alternative embodiment is characterized in that the first ends of the first longitudinal ribs are arranged on a first circular path and the second ends of the second longitudinal ribs are arranged on a second circular path and the two circular paths are arranged concentrically with respect to the longitudinal axis. Hence, as seen over the circumference, a uniform radial prestressing force of the protective cover onto the retaining protuberance of the base body can be established.

A further possible and optionally alternative embodiment has the features that a difference value of the first radial distance minus the second radial distance is in a difference value range the lower limit of which is 0.01 mm, preferably 0.04 mm, and the upper limit of which is 0.1 mm, preferably 0.06 mm. Thus, depending on the selected difference value, the possible tolerance compensation can be determined. Moreover, hence, the holding force on the base body and the required axial pull-off force can be determined in certain limits.

In a further embodiment, it is provided that the total number of the first longitudinal ribs and the second longitudinal ribs is an even number and is selected from a total number of four, six, eight or ten. Thus, the protective cover can be easily adapted to different conditions of use and dimensions.

Another embodiment is characterized in that the first longitudinal ribs and/or second longitudinal ribs, as seen in radial section, each have a wave shape. Thus, erratic transitions in wall thickness can be avoided.

For the purpose of better understanding of the invention, it will be elucidated in more detail by means of the figures below.

These show in a respectively very simplified schematic representation:

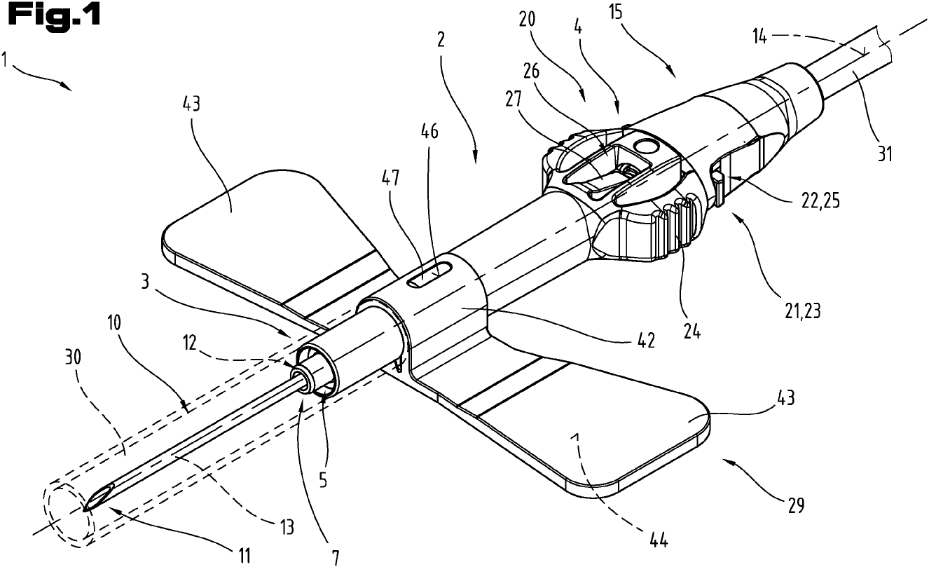

FIG. 1 a cannula arrangement in its unused operational position in a graphic representation;

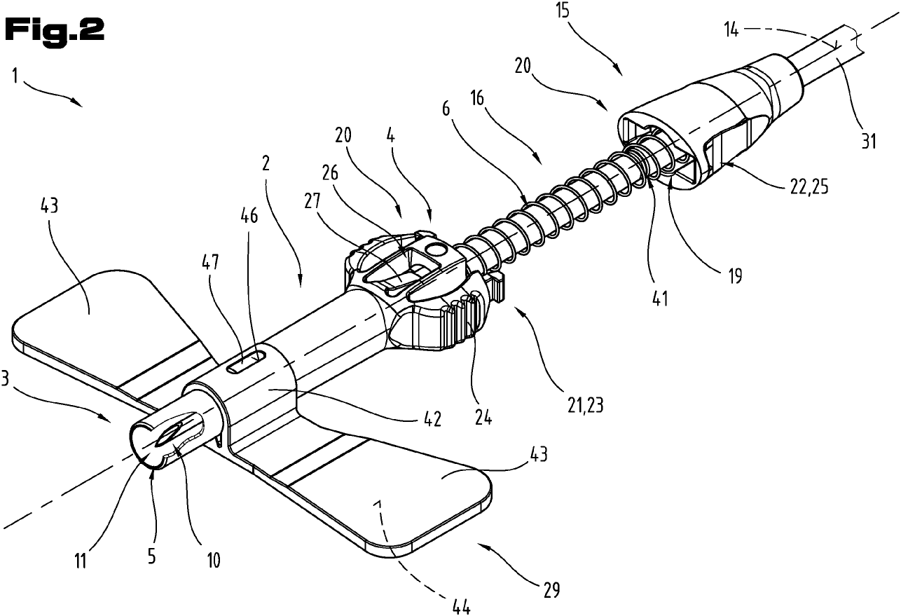

FIG. 2 the cannula arrangement according to FIG. 1 but in its protective position of the cannula, in a graphic representation, in a partially sectional view;

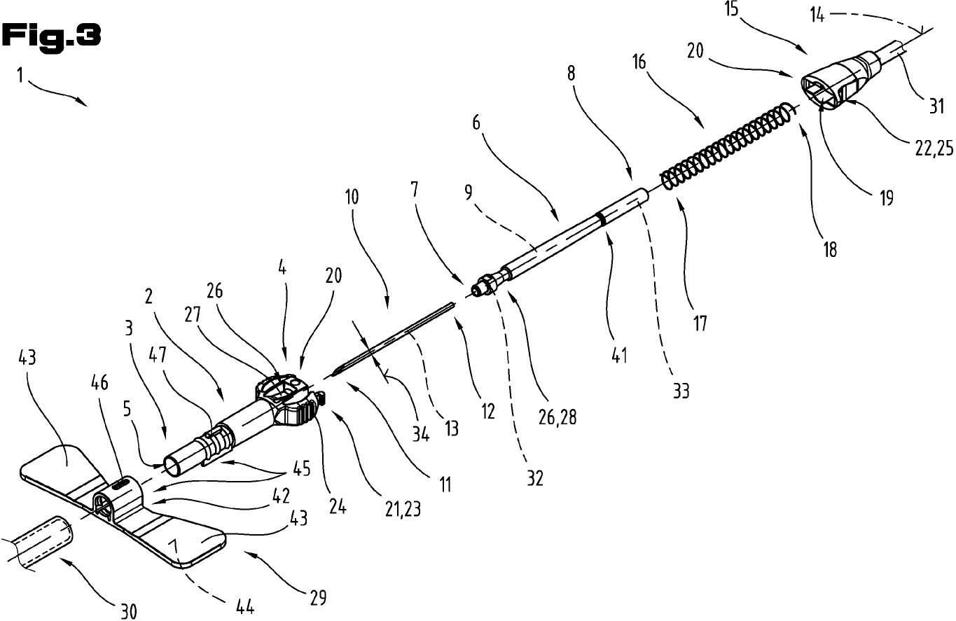

FIG. 3 the cannula arrangement according to FIGS. 1 and 2 in a kind of exploded view with individual components distanced from each other;

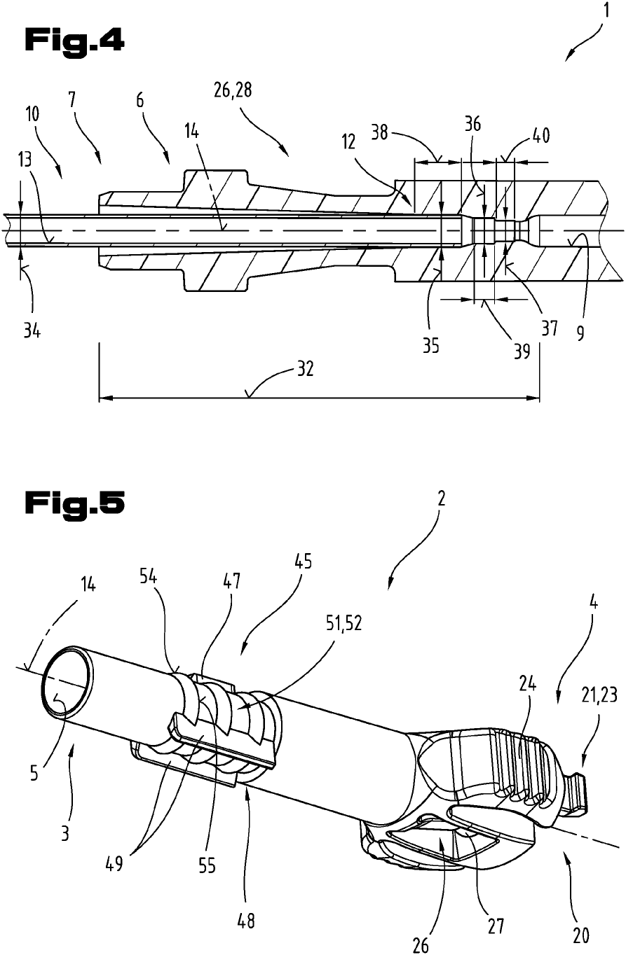

FIG. 4 the cannula support with the cannula held thereon, in axial section and in an enlarged view;

FIG. 5 the base body of the cannula arrangement according to FIGS. 1 to 4 in a graphic representation;

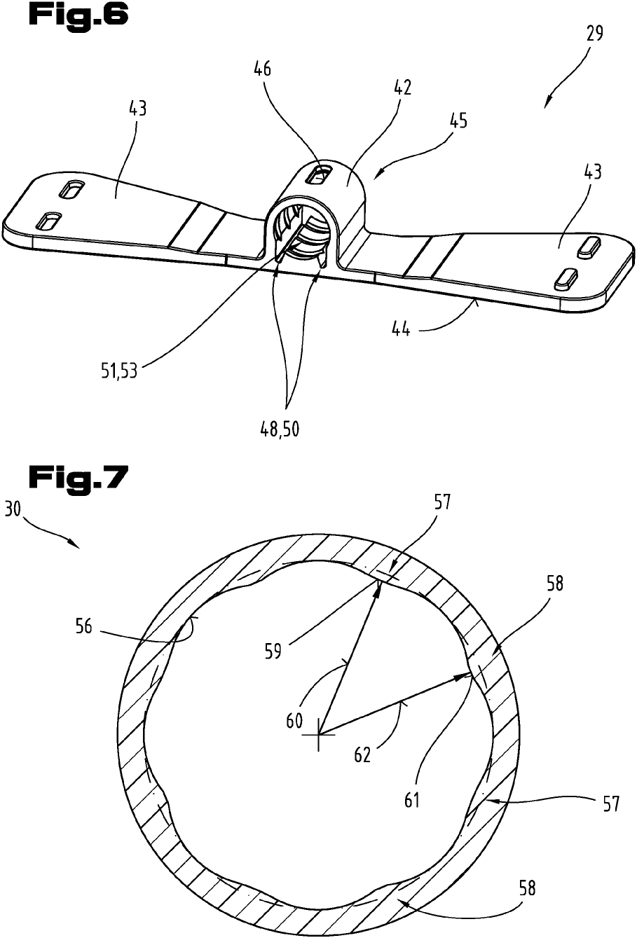

FIG. 6 the wing arrangement of the cannula arrangement according to FIGS. 1 to 5 in a graphic representation;

FIG. 7 a possible cross-section of the protective cover of the cannula arrangement in an enlarged view;

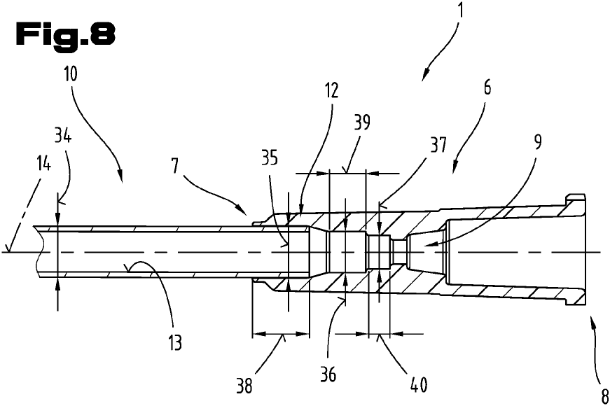

FIG. 8 another cannula support embodied as a female Luer coupling part with a cannula held thereon, in axial section.

First of all, it is to be noted that in the different embodiments described, equal parts are provided with equal reference numbers and/or equal component designations, where the disclosures contained in the entire description may be analogously transferred to equal parts with equal reference numbers and/or equal component designations. Moreover, the specifications of location, such as at the top, at the bottom, at the side, chosen in the description refer to the directly described and depicted figure and in case of a change of position, these specifications of location are to be analogously transferred to the new position.

The term “in particular” shall henceforth be understood to mean that it may refer to a possible more specific formation or more detailed specification of an object or a process step, but need not necessarily depict a mandatory, preferred embodiment of same or a mandatory practice.

Below, the terms “distal” and “proximal” will be used. The positional indication or directional indication by the term “distal” shall be interpreted to mean that this end or end section of the respective object faces away from the user thereof and can be turned towards a patient. The further positional indication or directional indication by the term “proximal” shall be interpreted to mean that this end or end section of the respective object faces the user thereof and faces away from the patient.

FIGS. 1 to 7 show a cannula arrangement 1 which is, in general, used in medical technology. Such cannula arrangements 1 can also be referred to as safety cannula arrangement or as safety needle arrangement. The cannula arrangement 1 can, for example, be used to remove fluid, in particular blood or another body fluid, from a body and/or to supply fluid into a body. The shown cannula arrangement 1 can also be referred to as a so-called “butterfly” which is usually held on the body during intended use. This can, for example, be done by means of an adhesive tape, as is sufficiently known. FIGS. 1 and 2 show a cannula arrangement 1 in its assembled state, but in different positions of its individual component parts with respect to one another.

FIG. 3 shows the individual components and/or component parts of the cannula arrangement 1 in an arrangement where they are separated from one another to provide a better overview and possibility of description of their embodiment.

In the present exemplary embodiment, the cannula arrangement 1 comprises a base body 2 with a distal end 3 and a proximal end 4. Preferably, the base body 2 is tube-shaped, in particular hollow-cylindrical, and has a hole 5 extending between the distal end 3 of the base body 2 and the proximal end 4 of the base body 2 in the direction of its axial extension.

Furthermore, the safety needle arrangement 1 can also comprise a cannula support 6, which, in turn, has a distal end 7 and a proximal end 8 distanced therefrom in the axial direction. In the cannula support 6, a flow-through channel 9 extends between the distal end 7 and the proximal end 8. Moreover, the cannula support 6 is embodied such that it is received in the hole 5 of the base body 2 and is guided therein being adjustable in the axial direction.

Furthermore, the cannula arrangement 1 can also comprise a cannula 10, which, in turn, has a distal end 11 and a proximal end 12 distanced therefrom in the axial direction. Within the cannula 10, a flow channel 13 extends between the distal end 11 and the proximal end 12. Moreover, the proximal end 12 of the cannula 10 can be held on the distal end 7 of the cannula support 6, in particular be fixedly attached thereto. The connection of the proximal end 12 of the cannula 10 to the cannula support 6 can, for example, be established by an at least air-tight bonding connection. However, irrespectively thereof it would also be possible to form a force fit instead of the bonding connection or additionally thereto. For this purpose, a corresponding measuring difference between the end region of the cannula 10 and the receiving opening in the cannula support 6 is to be formed. The cannula support 6 and the cannula 10 held thereon are preferably aligned coaxially to one another and define a longitudinal axis 14 together.

Hence, starting out from the distal end 11 of the cannula 10, a flow of fluid through the flow channel 13 and, further, through the flow-through channel 9 in the cannula support 6 or in the opposite direction can be achieved.

Furthermore, the cannula support 6 can also have an additional retaining protuberance 15 in the region of its proximal end 8. The retaining protuberance 15 can be formed either by a separate components or be an integral part of the cannula support 6. For example, if the components are each separate, as in the present exemplary embodiment, the proximal end 8 of the cannula support 6 can extend into the retaining protuberance 15, as seen in the axial direction, and is connected to it there. Here too, the mutual mounting and/or connection between the proximal end 8 of the cannula support 6 and the retaining protuberance 15 can be achieved, for example, by an at least air-tight bonding connection. However, irrespectively thereof it would also be possible to form a force fit instead of the bonding connection or additionally thereto. For this purpose, a corresponding measuring difference between the end region of the cannula support 6 and the retaining protuberance 15 is to be formed.

For automatic displacement of the cannula support 6 relative to the base body 2, the cannula arrangement 1 can also comprise a separate adjusting element 16, which is arranged to act between the base body 2 and the cannula support 6 in the installed state. In the present exemplary embodiment, the adjusting element 16 is formed by a spiral compression spring, which is arranged extending on the outer side of the cannula support 6. A distal adjusting element end 17 facing the base body 2 is supported thereon in the region of the proximal end 4 of the base body 2. The adjusting element 16 can also extend in the axial direction over a partial length into the base body 2.

In the present exemplary embodiment, the further proximal adjusting element end 18 of the adjusting element 16 distanced therefrom in the axial direction is supported on the retaining protuberance 15. In order to have sufficient space for the adjusting element 16 in the slid-together operational position and/or pretensioned position, it can be advantageous if the adjusting element 16 can extend over a partial length on the side facing the base body 2 into the retaining protuberance 15. For this purpose, a receiving opening 19 for receiving the adjusting element 16, which extends across a partial length between the cannula support 6 and the retaining protuberance 15, is arranged and/or provided in the retaining protuberance 15 on its side facing the base body 2. In this regard, the cross-sectional shape of the receiving opening 19 is preferably to be adapted to the dimensions of the adjusting element 16. In the present exemplary embodiment, the receiving opening 19 is embodied, for example, tube-shaped and/or hollow-cylindrically.

FIG. 1 shows the first relative position of the cannula support 6 together with the cannula 10 held thereon. In this first position, the cannula 10 projects over the distal end 3 of the base body 2.

FIG. 2 shows the so-called protective position, which is referred to as second position here. In this second position, at least the distal end 11 of the cannula 10 is covered by the base body 2 to thus prevent puncture wounds by the used cannula 10. The displacement, in particular the axial displacement, of the cannula support 6 together with the cannula 10 held thereon is carried out automatically by the adjusting element 16 described above. If the base body 2 is considered stationarily positioned, the relative displacement of the cannula support 6 together with the cannula 10 takes place in the proximal direction.

In order to hold the cannula support 6 together with the cannula 10 held thereon locked and/or arrested in the first position relative to the base body 2, a separate retaining device 20 is provided, which also can be part of the cannula arrangement 1. In this exemplary embodiment, the retaining device 20 comprises multiple first retaining elements 21 as well as multiple second locking elements 22. In the retaining and/or locking position of the retaining device 20, the first relative position of the cannula support 6 together with the cannula 10 relative to the base body 2 is determined by the engaged first retaining elements 21 and second retaining elements 22.

In the present exemplary embodiment, the first retaining elements 21 are formed by diametrically opposed projections 23 and are arranged or formed in the region of the proximal end 4 of the base body 2 thereon. Moreover, the projections 23 can each be arranged on a retaining arm 24 adjustable in the radial direction.

In the present exemplary embodiment, the second retaining elements 22 are formed by recesses 25 which are also arranged diametrically opposite one another to allow for engagement with the first retaining elements 21 of the retaining device 20 described above. In this regard, the second retaining elements 22 are arranged in the region of the proximal end 8 of the cannula support 6. In the present exemplary embodiment, the recesses 25 forming the second retaining elements 22 are arranged or formed in the retaining protuberance 15.

By this radial adjustment and/or displacement of the projections 23 in each case arranged on a retaining arm 24, these disengage from the recesses 25, whereby the retaining device 20 disengages. As soon as the engagement of the retaining device 20 has been released, the adjusting element 16 comes into action and self-actingly and automatically moves the cannula support 6 and thus also the cannula 10 into its covered position (second position or protective position).

To avoid re-use of the cannula arrangement 1, the cannula support 6 may be retained in its second relative position with respect to the base body 2 by means of an arresting device 26 arranged in the region of the proximal end 4 of the base body 2, being locked to the base body in the axial direction relative thereto. This arresting device 26 can be embodied such that in the second position of the cannula support 6 it is hindered in terms of a repeated adjustment in both directions, as seen in the axial direction. Thus, it can be prevented that the cannula support 6 detaches from the base body 2 and simultaneously a repeated return of the cannula support 6 to the first position is prevented. For this purpose, multiple first arresting elements 27 and preferably multiple second arresting elements 28 are provided. The first arresting elements 27 are arranged in the region of the proximal end 4 of the base body 2 and the second arresting elements 28 are arranged in the region of the distal end 7 of the cannula support 6.

Furthermore, a wing arrangement 29 projecting from the base body 2 in the radial direction can be arranged or formed thereon. The wings of the wing arrangement 29 can be in a position abutting on one another during the piercing operation for better support by the operator. When the cannula arrangement 1 together with its cannula 10 is pierced into the body for removing fluid from the body or for supplying fluids into the body, the entire cannula arrangement 1 with the wings of the wing arrangement 29 can be held and/or fixed to the surface of the body, which is not shown in greater detail, with an additional adhesive strip or another holding means.

In its unused state, the cannula arrangement 1 is usually packaged in a sterile packaging and is removed therefrom by an operator. In this regard, the cannula support 6 together with the cannula 10 held thereon is in the first position, in which the cannula 10 projects beyond the distal end 3 of the base body 2. To prevent a puncture wound in this state, FIG. 1 shows an additional, preferably tube-shaped, protective cover 30, which is indicated in dashed lines. The protective cover 30 can be formed by just a tube-shaped component open on both ends, wherein then the entire safety needle arrangement 1 is sterilely packaged in an additional overpack, e.g. a blister pack not shown, until it is used as intended. After removal of the protective cover 30 or the protective cap from the base body 2, the cannula 10 is exposed and can be inserted into the body at the intended puncture site.

Furthermore, it is indicated that, at the proximal end of the retaining protuberance 15, a hose 31 for establishing a connection to a further medial component can be arranged.

FIG. 4 shows a cannula support 6 with the cannula 10 attached thereto in an axial section, to provide a better view of the mounting of the cannula 10 within the flow-through channel 9 in the cannula support 6.

The flow-through channel 9, further, itself defines a distal flow-through channel end section 32 and a proximal flow-through channel end section 33. The cannula 10 is received and held in the distal flow-through channel end section 32. This can be done in particular by means of a bonding connection.

Depending on the purpose of use and/or medical requirements to be performed, cannulas 10 with cross-sectional cannula dimensions 34 differing from one another are required. These can, for example, have different outer diameters, wherein some exemplary outer dimensions are listed below: 0.5 mm / 0.6 mm / 0.7 mm / 0.8 mm / 0.9 mm.

In this cannula support 6, it is now provided that, as seen in axial section, the distal flow-through channel end section 32, starting from the distal end 7 of the cannula support 6 in the direction towards the proximal end 8 of the cannula support 6, has a cross-sectional shape of stepped design with a first cross-sectional dimension 35 and at least one further cross-sectional dimension 36, 37. In the present exemplary embodiment, three cross-sectional dimensions 35, 36, 37 differing from one another are provided, this being just an example. However, it is also possible that more or less than three cross-sectional dimensions differing from one another are provided. This also applies to the receiving sections 38, 39, 40 described below.

Each one of the individual cross-sectional dimensions 35, 36, 37, in turn, defines its own receiving section 38, 39, 40. These can be referred to as first, second and third receiving analogously to the cross-sectional dimensions. In each one of the different receiving sections 38, 39, 40, the respective cannula 10 with the corresponding cross-sectional cannula dimension 34 can be received.

The cross-sectional dimension 35, 36, 37 of each receiving section 38, 39, 40, starting out from the distal end 7 of the cannula support 6 in the direction towards the proximal end 8 of the cannula support 6, is in each case smaller than the cross-sectional dimension 37, 36, 35 of the receiving section 40, 39, 38 immediately upstream in the axial direction. The first receiving section 38 is located upstream of the second receiving section 39 and the latter, if present, is located upstream of the third receiving section 40. Preferably, the receiving sections 38, 39, 40 are each formed having a hollow-cylindrical cross-section.

Each one of the cross-sectional dimensions 35, 36, 37 of the receiving sections 38, 39, 40 differing from one another is embodied, in each case, to receive the cannula 10 with the corresponding cross-sectional cannula dimension 34. Thus, it is achieved that in each of the cross-sectional dimensions 35, 36, 37 of the receiving sections 38, 39, 40, merely the respective corresponding cannula 10 with its cross-sectional cannula dimension can be received, since the respective cross-sectional dimension 35, 36, 37 of the respective receiving section 38, 39, 40 is coordinated with and adapted to the respective cannula 10 with its cross-sectional cannula dimension to be received and held therein. Usually, the cross-sectional cannula dimensions are predetermined depending on the later purpose of use, wherein the individual cross-sectional dimensions 35, 36, 37 of the receiving sections 38, 39, 40 that differ from one another are selected such that the respective cannula 10 is inserted into the provided receiving section 38, 39, 40 and is or will be optionally also held and attached therein by means of an additional adhesive or bonding agent. For the sake of simplicity, merely one reference number is used for the cannula 10 with the respective cross-sectional cannula dimensions 34 differing from one another, since merely one single dimension is depicted. In the present exemplary embodiment, the cannula 10 with the cross-sectional cannula dimension 34 corresponding to the first cross-sectional dimension 35 of the first receiving section 38 is shown.

Each one of the receiving sections 38, 39, 40 on its proximal end forms an axial stop for the proximal end 12 of the cannula 10 held therein in each case. A relative positioning of the cannula 10 in the respective receiving section 38, 39, 40 predetermined in the axial direction is achieved when the respective proximal end 12 of the cannula 10 is supported against the corresponding axial stop of the respective receiving section 38 or 39 or 40 in the direction towards the proximal end 8 of the cannula support 6.

In the present exemplary embodiment, the cannula support 6 supporting and/or holding the cannula 10 is a hollow-cylindrical component part of a safety cannula arrangement. However, independently thereof, it would be possible to hold the cannula 10 on a cannula support 6 embodied as a female Luer coupling part, as is shown in a simplified manner in FIG. 8 below.

As described above, the arresting device 26 with the cooperating first and second arresting elements 27, 28 serves for locking the cannula support 6, when it is in the second position, in its relative position with respect to the base body 2 such that it cannot be displaced. This serves to prevent a reuse and/or unwanted puncture wounds.

The first arresting elements 27 of the arresting device 26 are embodied as arresting arms mounted on one side, which cooperate in a known manner with the annular surface designed as a circumferentially continuous surface (corresponding to the second arresting elements 28). At least one relief recess 41 is arranged or formed in the cannula support 6 to prevent material fatigue and the associated reduction of the restoring force in a preloaded position of the first arresting elements 27. The relief recess 41 can best be seen in FIG. 3. Hence, it is possible that when the cannula support 6 is in the first position, the first arresting elements 27 in their undeformed position project into the at least one relief recess 41. The relief recess 41 can, for example, be realized by a preferably circumferential groove-shaped recess or by a frustoconical or wedge-shaped partial section of the cannula support 6.

FIGS. 5 and 6 show and describe the wing arrangement 29 and its mounting and/or attachment to the base body 2 in further detail. The wing arrangement 29, in turn, comprises a predominantly tube-shaped retaining body 42 as well as wings 43 projecting from the retaining body 42 on both sides. Moreover, a coupling unit 45 on a pure mechanical basis can be arranged or formed between the inside of the retaining body 42 and the outside of the base body 2. By means of the coupling unit 45, the wing arrangement 29 is held in a coupling position on the base body 2.

To make a piercing success optically recognizable, at least the base body 2 and the cannula support 6 are made of a translucent or transparent material, usually a plastic material. When blood exits the proximal end 12 of the cannula 10, this can be optically recognized. It is additionally provided here that an observation window 46 is arranged in the tub-shaped retaining body 42 of the wing arrangement 29 on a side facing away from the contact side 44. The observation window 46 is fully surrounded by material of the retaining body 42 and fully penetrates the retaining body 42 in the radial direction and thus in the direction towards the longitudinal axis 14.

It can be seen from a combination of FIGS. 1, 2, 5 and 6 that a protuberance 47 projecting in the radial direction is arranged or formed on the base body 2, said protuberance 47 projecting into the observation window 46 when the wing arrangement 29 is in the coupling position on the base body 2.

To inhibit or entirely prevent a relative rotation and/or pivoting of the wing arrangement 29 with respect to the base body 2 about the longitudinal axis 14, the coupling unit 45 further comprises an anti-rotation device 48 with at least one first anti-rotation element 49 on or in the base body 2 and at least one second anti-rotation element 50 cooperating therewith in or on the retaining body 42. In the present exemplary embodiment, the first anti-rotation element 49 or the first anti-rotation elements 49 is/are embodied as a web(s). The second anti-rotation element 50 is or the second anti-rotation elements 50 are designed as a groove(s). The anti-rotation elements 49, 50 each have a parallel longitudinal alignment with respect to the longitudinal axis 14. The respective cooperating anti-rotation elements 48, 50, as seen in radial section and with horizontal alignment of the contact side 44, which usually defines a plane, are arranged or formed on both sides of a vertical plane and spaced apart from each other. The respective cooperating anti-rotation elements 48, 50 are embodied so as to be mirror-inverted.

In order to also prevent a separating movement of the wing arrangement 29 from the base body 2 that is aligned in the opposite direction to the push-on movement that takes place in the proximal direction, an axial securing device 51 with at least one first axial securing element 52 on or in the base body 2 and at least one second axial securing element 53 cooperating therewith in or on the retaining body 42 is provided. The axial securing device 51 can be considered a part of the coupling unit 45.

The base body 2 forms a stop, which can be formed by a bulge of the base body 2, acting in the axial direction for the proximal end of the retaining body 42. In the present exemplary embodiment, the first axial securing elements 52 are embodied approximately with a triangular cross-section as seen in axial section. The first boundary line 54 of the triangular cross-section, as viewed in the axial push-on direction of the wing arrangement 29 onto the base body 2, defines a ramp formed to rise on the side facing away from the longitudinal axis 14. A second boundary line 55 of the triangular cross-section is preferably oriented to run in a normal plane with respect to the longitudinal axis 14. The second axial securing elements 53 are to be embodied mirror-invertedly with respect to the first axial securing elements 52. Preferably, the first and second axial securing elements 52, 53 are formed and/or arranged to be continuous across the circumference and can be interrupted by the anti-rotation elements 49, 50 described above.

FIG. 7 shows a possible cross-section of the predominantly tubular or hollow-cylindrical protective cover 30, which can also be referred to as protective sleeve. The protective cover 30 serves for being arranged on the distal end 3 of the base body 2 and being detachably held thereon. Preferably, the protective cover 30 is made of a plastic material in an extrusion process and the individual protective covers 30 are separated from the otherwise endless tube body with the predetermined construction lengths. In general, a continuous cover wall having a sufficient inherent stiffness is formed. The cover wall without the longitudinal ribs 57, 58 that will be described below can, for example, have a wall thickness in a range with a lower limit of 0.25 mm and an upper limit of 0.35 mm, preferably of 0.3 mm. The ideal, undeformed wall thickness of the cover wall is indicated on the inner side with a dot-dashed line.

In the first position of the cannula support 6, the protective cover 30 covers the cannula 10 that projects from the base body 2 in the distal direction. In order to achieve, on the one hand, a sufficient holding and/or adhesive force of the protective cover 30 on the base body 2 and, on the other hand, a pull-off force of the protective cover 30 from the base body 2 defined within certain limits, multiple first longitudinal ribs 57 and second longitudinal ribs 58 distributed over the circumference are formed or provided on an inner surface 56 of the cover wall of the protective cover 30. In each case, at least two first longitudinal ribs 57 and at least two second longitudinal ribs 58 are provided. The longitudinal ribs 57 and 58 that are arranged distributed across the circumference are each arranged alternately to one another, i.e. a first longitudinal rib 57 and then a second longitudinal rib 58 and so on.

When the cross-section of the protective cover 30 is undeformed, the first ends 59 of the first longitudinal ribs 57 each end at a first radial distance 60 in front of the longitudinal axis 14. Likewise, when the cross-section of the protective cover 30 is undeformed, second ends 61 of the second longitudinal ribs 58 end at a second radial distance 62 in front of the longitudinal axis 14. The first radial distance 60 is larger than the second radial distance 62, wherein the second ends 61 of the second longitudinal ribs 58 are closer to the longitudinal axis 14. Since each of the first radial distances 60 and each of the second radial distances 62 are equal in size, the first ends 59 of the first longitudinal ribs 57 are in each case on a first circular path and the second ends 61 of the second longitudinal ribs 58 are in each case on a second circular path. The two circular paths are arranged concentrically with respect to the longitudinal axis 14. A difference value of the first radial distance 60 minus the second radial distance 62 is selected from a difference value range the lower limit of which is 0.01 mm, preferably 0.04 mm and the upper limit of which is 0.1 mm, preferably 0.06 mm. A preferred difference value can, for example, also be 0.05 mm. The total number of the first longitudinal ribs 57 and the second longitudinal ribs 58 is an even number and is selected from a total number of four, six, eight or ten. Preferably, the first longitudinal ribs 57 and/or the second longitudinal ribs 58 can each have a wave shape and/or wave-shaped transitions as seen in radial section.

Due to the radial distances 60 and 62 that are selected differently from one another and the alternate arrangement across the circumference, production tolerances in the protective cover 30 and the base body 2 can be better compensated for with respect to each other and the holding and/or adhesive force described above and the required pull-off force can still be realized and/or maintained within predetermined limits. Preferred pull-off forces are in a force range with a lower limit of 0.5 N, preferably 1 N and an upper limit of 10 N, preferably 4 N.

In the mounted state of the protective cover 30 on the protuberance of the base body 2 provided for this purpose, said protuberance having a cylindrical outer surface, the second longitudinal ribs 58 are in contact with the outer surface due to the smaller second radial distance 62. Depending on the achieved production tolerances, the first longitudinal ribs 57 with their first ends 59 can also be brought more or less into contact with the outer surface of the protuberance of the base body 2 due to the elastic properties of the plastic material of the protective cover 30 and the larger first radial distance 60. The circumferential wall of the protective cover 30 is deformed in the region of the first longitudinal ribs 57 from its ideal circular shape to an arc section with an arc radius larger than the ideal circular shape. Hence, in this arc section of the first longitudinal ribs 57, the otherwise built-up radial force in the direction towards the longitudinal axis 14 can be reduced within certain limits. If, for example, there are four first longitudinal ribs 57 and the four second longitudinal ribs 58 between these, an approximate 4-corner alignment is achieved in the mounted state.

Furthermore, it can be provided that for an easier sliding movement of the cannula support 6 within the hole 5 if the base body 2, a coating is applied to at least one surface of the aforementioned components. Advantageously, the same coating with which the cannula 10 is coated can be used. Such a coating can, for example, be a silicone coating which is embodied to be cured after it has been applied to the substrate surface. The solvent contained in the coating agent evaporates after the coating process and the remaining coating agent bonds with the carrier substrate to which it has been applied.

The exemplary embodiments show possible embodiment variants, and it should be noted in this respect that the invention is not restricted to these particular illustrated embodiment variants of it, but that rather also various combinations of the individual embodiment variants are possible and that this possibility of variation owing to the technical teaching provided by the present invention lies within the ability of the person skilled in the art in this technical field.

The scope of protection is determined by the claims. Nevertheless, the description and drawings are to be used for construing the claims. Individual features or feature combinations from the different exemplary embodiments shown and described may represent independent inventive solutions. The object underlying the independent inventive solutions may be gathered from the description.

All indications regarding ranges of values in the present description are to be understood such that these also comprise random and all partial ranges from it, for example, the indication 1 to 10 is to be understood such that it comprises all partial ranges based on the lower limit 1 and the upper limit 10, i.e. all partial ranges start with a lower limit of 1 or larger and end with an upper limit of 10 or less, for example 1 through 1.7, or 3.2 through 8.1, or 5.5 through 10.

Finally, as a matter of form, it should be noted that for ease of understanding of the structure, elements are partially not depicted to scale and/or are enlarged and/or are reduced in size.

LIST OF REFERENCE NUMBERS

| | | | |
|---|---|---|---|
| 1 | Cannula arrangement | 32 | Distal flow-through channel end section |
| 2 | Base body | 33 | Proximal flow-through channel end section |
| 3 | Distal end | 34 | Cross-sectional cannula dimension |
| 4 | Proximal end | 35 | First cross-sectional dimension |
| 5 | Hole | 36 | Second cross-sectional dimension |
| 6 | Cannula support | 37 | Third cross-sectional dimension |
| 7 | Distal end | 38 | First receiving section |
| 8 | Proximal end | 39 | Second receiving section |
| 9 | Flow-through channel | 40 | Third receiving section |
| 10 | Cannula | 41 | Relief recess |
| 11 | Distal end | 42 | Retaining body |
| 12 | Proximal end | 43 | Wing |
| 13 | Flow channel | 44 | Contact side |
| 14 | Longitudinal axis | 45 | Coupling unit |
| 15 | Retaining protuberance | 46 | Observation window |
| 16 | Adjusting element | 47 | Protuberance |
| 17 | Distal adjusting element end | 48 | Anti-rotation device |
| 18 | Proximal adjusting element end | 49 | First anti-rotation element |
| 19 | Receiving opening | 50 | Second anti-rotation element |
| 20 | Retaining device | 51 | Axial securing device |
| 21 | First retaining element | 52 | First axial securing element |
| 22 | Second retaining element | 53 | Second axial securing element |
| 23 | Projection | 54 | First boundary line |
| 24 | Retaining arm | 55 | Second boundary line |
| 25 | Recess | 56 | Inner surface |
| 26 | Arresting device | 57 | First longitudinal rib |
| 27 | First arresting element | 58 | Second longitudinal rib |
| 28 | Second arresting element | 59 | First end |
| 29 | Wing arrangement | 60 | First radial distance |
| 30 | Protective cove | 61 | Second end |
| 31 | Hose | 62 | Second radial distance |

1. A cannula arrangement (1), in particular a safety cannula arrangement, comprising
a cannula support (6) with a distal end (7) and a proximal end (8), wherein a flow-through channel (9) extends within the cannula support (6) between its distal end (7) and its proximal end (8), and the flow-through channel (9) defines a distal flow-through channel end section (32) and a proximal flow-through channel end section (33),
a cannula (10) with a distal end (11) and a proximal end (12), wherein a flow channel (13) extends within the cannula (10) between its distal end (11) and its proximal end (12), and
wherein the proximal end (12) of the cannula (10) is received in the distal flow-through channel end section (32) of the cannula support (6) and is held on the cannula support (6), in particular glued therewith, and the cannula (10) and the cannula support (6) de-fine a longitudinal axis (14),
wherein as seen in axial section, the distal flow-through channel end section (32), starting from the distal end (7) of the cannula support (6) in the direction towards the proximal end (8) of the cannula support (6), has a cross-sectional shape of stepped design with a first cross-sectional dimension (35) and at least one further cross-sectional dimension (36, 37),
wherein each one of the cross-sectional dimensions (35, 36, 37) defines an own receiving section (38, 39, 40) for the respective cannula (10) to be received therein,
wherein the cross-sectional dimension (35, 36, 37) of each receiving section (38, 39, 40), starting from the distal end (7) of the cannula support (6) in the direction towards the proximal end (8) of the cannula support (6), is in each case smaller than the cross-sectional dimension (37, 36, 35) of the receiving section (40, 39, 38) immediately upstream in the axial direction, and
wherein each one of the cross-sectional dimensions (35, 36, 37) of the receiving sections (38, 39, 40), which differ from one another, is embodied to receive the cannula (10) with a corresponding cross-sectional cannula dimension (34) in each case.

2. The cannula arrangement (1) according to claim 1, wherein the receiving sections (38, 39, 40) are embodied so as to each have a hollow-cylindrical cross-section.

3. The cannula arrangement (1) according to claim 1, wherein multiple, in particular three, receiving sections (38, 39, 40) formed behind one another in the axial direction are provided.

4. The cannula arrangement (1) according to claim 1, wherein each one of the receiving sections (38, 39, 40) on its proximal end forms an axial stop for the proximal end (12) of the respective cannula (10) received therein in each case.

5. The cannula arrangement (1) according to claim 4, wherein the respective proximal end (12) of the cannula (10) is supported on the corresponding axial stop of the respective receiving section (38, 39, 40) resting thereon the direction towards the proximal end (8) of the cannula support (6).

6. The cannula arrangement (1) according to claim 1, wherein the cannula support (6) is formed by a female Luer coupling part or a component part of a safety cannula arrangement.

7. The cannula arrangement (1) according to claim 1, wherein, furthermore, a base body (2) with a distal end (3) and a proximal end (4) is provided, wherein a hole (5) extends in the base body (2) between the distal end (3) of the base body (2) and the proximal end (4) of the base body (2), and wherein at least the cannula support (6) with the cannula (10) held thereon is received in the hole (5) and is adjustable in the axial direction in the hole (5).

8. The cannula arrangement (1) according to claim 1, wherein, furthermore, an automatically acting adjusting element (16) is provided, said adjusting element (16) being arranged acting between the base body (2) and the cannula support (6), and the adjusting element (16) displaces the cannula support (6) together with the cannula (10) held thereon from a first position, in which first position the cannula (10) projects beyond the distal end (3) of the base body (2), into a second position, in which second position at least the distal end (11) of the cannula (10) is covered by the base body (2).

9. The cannula arrangement (1) according to claim 1, wherein, furthermore, a retaining device (20) with multiple first retaining elements (21) and multiple second retaining elements (22) is provided, said retaining device (20) defining the first position of the cannula support (6) with respect to the base body (2) in the mutually engaged position of the first and second retaining elements (21, 22), and wherein the first retaining elements (21) are arranged in the region of the proximal end (4) of the base body (2) and the second retaining elements (22) are arranged in the region of the proximal end (8) of the cannula support (6).

10. The cannula arrangement (1) according to claim 1, wherein, furthermore, an arresting device (26) with multiple first and second arresting elements (27, 28) is provided, said arresting device (26) defining the second position of the cannula support (6) with respect to the base body (2) in the mutually engaged position of the first and second arresting elements (27, 28), and wherein the first arresting elements (27) are arranged in the region of the proximal end (4) of the base body (2) and the second arresting elements (28) are arranged in the region of the distal end (7) of the cannula support (6).

11. The cannula arrangement (1) according to claim 1, wherein at least one relief recess (41) is arranged or formed in the cannula support (6), and wherein, when the cannula support (6) is in the first position, the first arresting elements (27) project into the at least one relief recess (41) in their undeformed position.

12. The cannula arrangement (1) according to claim 1, wherein, furthermore, a wing arrangement (29) with a tube-shaped retaining body (42) and wings (43) projecting from the retaining body (42) on both sides is provided, wherein the wings (43) define a contact side (44) which can be positioned to face a patient, and wherein, furthermore, a coupling unit (45) is arranged or formed between the retaining body (42) and the base body (2), and the wing arrangement (29) is held in a coupling position on the base body (2) by means of the coupling unit (45).

13. The cannula arrangement according to claim 12, wherein an observation window (46) is formed in the tube-shaped retaining body (42), said observation window (46) being arranged on a side facing away from the contact side (44), and wherein in the coupling position, a protuberance (47) projecting from the base body (2) in the radial direction projects into the observation window (46).

14. The cannula arrangement (1) according to claim 12, wherein the coupling unit (45) further comprises an anti-rotation device (48) with at least one first anti-rotation element (49) on or in the base body (2) and at least one second anti-rotation element (50) cooperating therewith in or on the retaining body (42).

15. The cannula arrangement (1) according to claim 12, wherein the at least one first anti-rotation element (49) is embodied as a web and the at least one second anti-rotation element (50) is embodied as a groove, and wherein the anti-rotation elements (49, 50) have a parallel longitudinal alignment with respect to the longitudinal axis (14).

16. The cannula arrangement (1) according to claim 12, wherein the coupling unit (45) further comprises an axial securing device (51) with at least one first axial securing element (52) on or in the base body (2) and at least one second axial securing element (53) cooperating therewith in or on the retaining body (42).

17. The cannula arrangement (1) according to claim 16, wherein the axial securing elements (52, 53), as seen in axial section, are formed approximately with a triangular cross-section, wherein a first boundary line (54) of the triangular cross-section, as viewed in the axial push-on direction of the wing arrangement (29) onto the base body (2), defines a ramp formed to rise on the side facing away from the longitudinal axis (14), and a second boundary line (55) of the triangular cross-section is oriented to run in a normal plane with respect to the longitudinal axis (14).

18. The cannula arrangement (1) according to claim 1, wherein, furthermore, an in particular tube-shaped protective cover (30) is provided and the protective cover (30) is arranged on the distal end (3) of the base body (2) and is detachably held thereon, wherein the cannula (10) projecting from the base body (2) in distal direction in the first position is covered by the protective cover (30).

19. The cannula arrangement (1) according to claim 18, wherein at least two first longitudinal ribs (57) and at least two second longitudinal ribs (58) are arranged or formed distributed across the circumference on an internal surface (56) of the protective cover (30) facing the longitudinal axis (14), and wherein a first longitudinal rib (57) and a second longitudinal rib (58) are arranged alternately in the circumferential direction in each case.

20. The cannula arrangement (1) according to claim 19, wherein, in the undeformed cross-section of the protective cover (30), first ends (59) of the first longitudinal ribs (57) end at a first radial distance (60) in front of the longitudinal axis (14) and second ends (61) of the second longitudinal ribs (58) end at a second radial distance (62) in front of the longitudinal axis (14), wherein the first radial distance (60) is larger than the second radial distance (62).

21. The cannula arrangement (1) according to claim 20, wherein the first ends (59) of the first longitudinal ribs (57) are arranged on a first circular path and the second ends (61) of the second longitudinal ribs (58) are arranged on a second circular path and the two circular paths are arranged concentrically with respect to the longitudinal axis (14).

22. The cannula arrangement (1) according to claim 20, wherein a difference value of the first radial distance (60) minus the second radial distance (62) is in a difference value range the lower limit of which is 0.01 mm, preferably 0.04 mm, and the upper limit of which is 0.1 mm, preferably 0.06 mm.

23. The cannula arrangement (1) according to claim 19, wherein the total number of the first longitudinal ribs (57) and the second longitudinal ribs (58) is an even number and is selected from a total number of four, six, eight or ten.

24. The cannula arrangement (1) according to claim 19, wherein the first longitudinal ribs (57) and/or second longitudinal ribs (58), as seen in radial section, each have a wave shape.
